(19) United States
(12) Patent Application Publication
(10) Pub. No.: US 2013/0158418 A1
(43) Pub. Date: Jun. 20, 2013

(54) BLOOD PRESSURE MEASUREMENT APPARATUS AND BLOOD PRESSURE MEASUREMENT METHOD

(71) Applicant: Seiko Epson Corporation, Tokyo (JP)

(72) Inventor: Hiromitsu MIZUKAMI, Shiojiri (JP)

(73) Assignee: SEIKO EPSON CORPORATION, Tokyo (JP)

(21) Appl. No.: 13/690,486

(22) Filed: Nov. 30, 2012

(30) Foreign Application Priority Data
Dec. 14, 2011 (JP) 2011-273629
Oct. 12, 2012 (JP) 2012-226666

(51) Int. Cl. A61B 5/022; A61B 5/00; A61B 5/107

(52) U.S. Cl. CPC A61B 5/022; A61B 5/1075; A61B 5/7225
USPC 600/490

(57) ABSTRACT

In an ultrasound blood pressure monitor, a blood pressure diameter measurement section measures blood pressure diameter of a radial artery which is a measurement target based on reception results of ultrasound from an ultrasound sensor. In addition, a pressurizing section adds a pressure from a body surface so that the radial artery is pressed. Then, a correlation formula, which expresses a relationship between blood vessel diameter and blood pressure of the radial artery under pressurization by the pressurizing section, is found and stored in a storage section. Then, a blood pressure calculation section calculates blood pressure using the blood vessel diameter which is measured under pressurization and storage data in the storage section by controlling the pressurization operation of the pressurizing section.

### CROSS-REFERENCE TO RELATED APPLICATIONS

This application claims priority to Japanese Patent Application No. 2011-273629 filed on Dec. 14, 2011 and Japanese Patent Application No. 2012-226666 filed on Oct. 12, 2012. The entire disclosure of Japanese Patent Application Nos. 2011-273629 and 2012-226666 is hereby incorporated herein by reference.

### BACKGROUND

1. Technical Field

The present invention relates to an apparatus, which measures blood pressure of a patient, and the like.

2. Background Technology

From the related art, an apparatus which measures blood flow, blood vessel diameter, and blood pressure using ultrasound or the like and an apparatus which measures elasticity of a blood vessel have been proposed. These apparatuses have a characteristic in that measurement is possible without imparting pain or an unpleasant feeling to the patient.

For example, a technique is disclosed in Patent Document 1 where changes in blood pressure and changes in blood vessel diameter are assumed to have a non-linear relationship and blood pressure is calculated from a blood vessel elasticity index called a stiffness parameter and from the blood vessel diameter.

Japanese Laid-open Patent Publication No. 2004-41382 (Patent Document 1) is an example of the related art.

### SUMMARY

### Problems to Be Solved by the Invention

The technique which is disclosed in PTL1 is a technique where blood pressure is calculated based on correlation characteristics of blood vessel diameter and blood pressure. However, in an artery which is comparatively thin such as a limb artery, variation in the blood vessel diameter with regard to the change in blood pressure is extremely slight since the blood vessel is hard.

For example, in a radial artery which flows in the wrist, the change in blood vessel diameter is approximately 40 [μm] in relation to a change in blood pressure accompanying a pulsation which is approximately 50 [mmHg]. Accordingly, a blood vessel diameter measurement method, where measurement in units of a minimum of 8 [μm] is possible, is required in order to calculate blood pressure with, for example, a degree of accuracy of 10 [mmHg]. However, it is considered that a blood vessel diameter measurement method which realizes this degree of accuracy is difficult to realize.

The invention is carried out in consideration of the problems described above and has an advantage of proposing a novel technique for improving the degree of accuracy in blood pressure calculation.

### Means Used to Solve the Above-Mentioned Problems

A first embodiment which solves the problems above is a blood pressure measurement apparatus which is provided with a blood vessel diameter measurement section which measures blood vessel diameter of an artery which is a measurement target, a pressurizing section which adds pressure from a body surface so that the artery is pressed, a storage section which stores a relationship between blood vessel diameter and blood pressure of the artery under pressurization by the pressurizing section, and a blood pressure calculation section which calculates blood pressure by controlling a pressurization operation of the pressuring section using the blood vessel diameter, which is measured by the blood vessel diameter measurement section under pressurization, and storage data in the storage section.

In addition, as another embodiment, a blood vessel diameter measurement method for a blood vessel diameter measurement apparatus which is provided with a pressurizing section which adds pressure from a body surface so that an artery which is a measurement target is pressed and a storage section which stores the relationship between blood vessel diameter and blood pressure of the artery under pressurization by the pressurizing section, the method including measuring blood vessel diameter of the artery, and calculating blood pressure by controlling a pressurization operation of the pressuring section using the blood vessel diameter under pressurization and storage data in the storage section.

According to the first embodiment, the blood vessel diameter of the artery which is the measurement target is measured. Along with this, the pressurizing section adds pressure from the body surface so that the artery is pressed. Then, the relationship between the blood vessel diameter and the blood pressure of the artery under pressurization by the pressurizing section is stored and the blood pressure is calculated by controlling the pressurization operation of the pressuring section using the blood vessel diameter which is measured under pressurization, and the storage data in the storage section. According to experiments which were performed by the present inventors, the variation width in the blood vessel diameter with regard to the same change in blood pressure increases when pressure is added from the body surface so that the artery is pressed compared to when there is no pressurization. Accordingly, it is possible to reduce the effect of blood vessel diameter measurement errors by using pressurization and it is possible to improve the degree of accuracy in the blood pressure calculation.

In addition, as a second embodiment, the blood pressure measurement apparatus in the blood pressure measurement apparatus of the first embodiment can be a configuration where there is further provided a first pressure search section which searches for a pressure by controlling the pressure due to the pressurizing section so as to change so that the variation width in the blood vessel diameter accompanying a pulsation, which is measured by the blood vessel diameter measurement section, satisfies a predetermined condition, the storage section stores the relationship between the blood vessel diameter and the blood pressure of the artery in a state of pressurization with the pressure which has been searched for by the first pressure search section, and the blood pressure calculation section controls the pressurization operation of the pressurizing section so that there is pressurization with the pressure which is searched for by the first pressure search section.

According to the second embodiment, the pressure is searched for by the first pressure search section by controlling the pressure due to the pressurizing section so as to change so that the variation width in the blood vessel diameter accompanying a pulsation, which is measured by the blood vessel diameter measurement section, satisfies the predetermined condition. On top of this, the relationship between the blood vessel diameter and the blood pressure of the artery, which is in a state of pressurization with the pressure which has been searched for by the first pressure search section, is stored in the storage section. The blood pressure calculation section controls the pressurization operation of the pressurizing section so that there is pressurization with the pressure which has been searched for by the first search section. The relationship between the blood vessel diameter and the blood pressure in a case of pressurization with the pressure which has been searched for is stored as data and it is possible to correctly calculate the blood pressure of a patient by calculating the blood pressure by referencing the blood vessel diameter which is measured in a state of pressurization with a pressure which is the same as this and the storage data in the storage section.

In addition, as a third embodiment, the blood pressure measurement apparatus in the blood pressure measurement apparatus of the second embodiment can be a configuration where the first pressure search section searches for a pressure where the variation width of the blood vessel diameter accompanying a pulsation, which is measured by the blood vessel diameter measurement section, exceeds a predetermined variation width threshold which is set based on the relationship between pulse pressure and the variation width.

According to the third embodiment, it is possible to adjust the pressure which is pressurized by the pressurizing section due to the first pressure search section searching for a pressure where the variation width of the blood vessel diameter accompanying a pulsation, which is measured by the blood vessel diameter measurement section, exceeds a predetermined variation width threshold which is set based on the relationship between pulse pressure and the variation width.

In addition, as a fourth embodiment, the blood pressure measurement apparatus in the blood pressure measurement apparatus of any of the first to the third embodiments can be a configuration where the blood pressure calculation section controls the pressurization operation of the pressurizing section so as to pressurize with an arbitrary pressure.

According to the fourth embodiment, the pressurization operation of the pressurizing section is controlled so as to pressurize with an arbitrary pressure at a time when calculating the blood pressure. It is possible to increase variation in the blood vessel diameter as the pressure increases and it is possible to improve the degree of accuracy in blood pressure calculation. Accordingly, it is possible to calculate the blood pressure with an arbitrary setting of the pressure in order to secure the degree of accuracy in blood pressure calculation.

In addition, as a fifth embodiment, the blood pressure measurement apparatus in the blood pressure measurement apparatus of the fourth embodiment can be a configuration where there is further provided a second pressure search section which searches for a pressure by controlling the pressurization by the pressurizing section so as to change so that the blood vessel diameter which is measured by the blood vessel diameter measurement section satisfies a predetermined stability condition, and the blood pressure calculation section controls the pressurization of the pressurizing section so that there is pressurization with the pressure which is searched for by the second pressure search section.

For example, there is a possibility that a considerable error is included in the blood pressure calculation results in a case where there is large dispersion in the blood vessel diameter which is measured in a state of pressurization with a certain pressure. Therefore, according to the fifth embodiment, a pressure is searched for by controlling the pressurization by the pressurizing section so as to change so that the blood vessel diameter which is measured by the blood vessel diameter measurement section satisfies a predetermined stability condition. Then, the blood pressure calculation section controls the pressurization operation of the pressurizing section so that there is pressurization with the pressure which has been searched for. Due to this, the pressurization force of the pressurizing section is adjusted at a time when calculating the blood pressure during normal measurement and it is possible to improve the degree of accuracy in blood pressure calculation.

### BRIEF DESCRIPTION OF THE DRAWINGS

Referring now to the attached drawings which form a part of this original disclosure:

FIG. 1A is a configuration diagram of a blood pressure measurement system, and FIG. 1B is a diagram illustrating a mounting state of an ultrasound blood pressure monitor;

FIG. 2 is a cross sectional diagram of a state where an ultrasound blood pressure monitor is mounted on a wrist;

FIG. 3 is experiment results illustrating a relationship between a pressurization force and blood vessel diameter variation width;

FIG. 4 is an explanatory diagram of correlation characteristics of blood vessel diameter and blood pressure;

FIG. 5 is a block diagram illustrating an example of a functional configuration of an ultrasound blood pressure monitor;

FIG. 6 is a flow chart illustrating the flow of a main process;

FIG. 7 is a flow chart illustrating the flow of a correction process;

FIG. 8 is a diagram illustrating an example of a configuration of a blood pressure measurement system according to a second embodiment;

FIG. 9 is a diagram illustrating changes in blood vessel diameter variation width;

FIG. 10 is a diagram for describing effects of a blood pressure measurement method in the second embodiment; and

FIG. 11 is a flow chart illustrating the flow of a second main process.

### DETAILED DESCRIPTION OF EXEMPLARY EMBODIMENTS

As an embodiment where the invention is applied, an embodiment of a blood flow measurement apparatus which measures blood pressure of a patient will be described with the wrist of a patient as a measurement target portion and an artery which is the measurement target as the radial artery. Here, naturally, the embodiments where it is possible for the invention to be applied are not limited to the embodiments described below.

### 1. Schematic Configuration

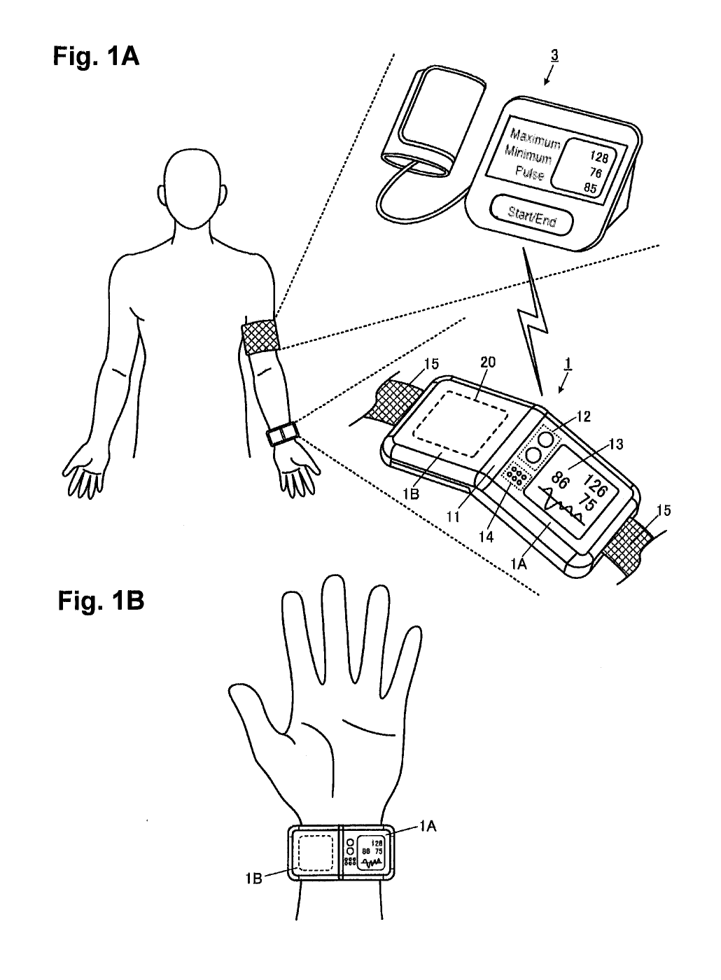

FIG. 1A is a configuration diagram of a system according to blood pressure measurement of the embodiment. The blood pressure measurement system is configured to have an ultrasound blood pressure monitor 1 which is configured to be able to be used by a patient mounting such on their wrist and a cuff type blood pressure monitor 3 which is used by being wrapped around the upper arm of a patient.

The cuff type blood pressure monitor 3 measures the blood pressure of an artery in the upper arm by the cuff which senses blood pressure being wrapped around the upper arm of the patient. In the embodiment, the cuff type blood pressure monitor 3 is used in order to perform correction of the ultrasound blood pressure monitor 1. After the correction has been performed, the cuff type blood pressure monitor 3 is removed and measurement of blood pressure is performed using the ultrasound blood pressure monitor 1 as a unit.

The ultrasound blood pressure monitor 1 is configured so that a body section is able to be mounted onto a measurement target portion (in particular, a wrist) of the patient using a strip section 15. The strip section 15 is a mounting tool for mounting an apparatus body onto the measurement target portion of the patient and is configured to have a band which is provided with a surface fastener, a clip which is for pinching a gauging section, and the like. The body section of the ultrasound blood pressure monitor 1 is configured to be connected to a first portion 1A and a section portion 1B via a hinge section 11.

An operation button 12, a liquid crystal display device 13, and a speaker 14 are provided in the first portion 1A.

The operation button 12 is used for the patient to operate and input starting instructions for the measurement of blood pressure and various types of amounts which are related to the measurement of blood pressure.

The blood pressure measurement results according to the ultrasound blood pressure monitor 1 are displayed in the liquid crystal display device 13. As the display method, a blood pressure measurement value can be displayed using a numerical value or there can be a display using a graph or the like.

There is audio output such as various types of voice guidance which are related to the measurement of blood pressure from the speaker 14. In the embodiment, measurement of blood pressure using the cuff type blood pressure monitor 3 is necessary in response to execution of a correction process. As a result, there can be audio output of voice guidance, which instructs the attaching and removal of the cuff type blood pressure monitor 3, from the speaker 14.

A sensor section 20 is provided in the second portion 1B. The sensor section 20 is configured to have an ultrasound sensor 21 and a pressurizing section 30.

The ultrasound sensor 21 is an ultrasound sending and receiving section where ultrasound oscillation units are lined up in an array formation. The ultrasound sensor 21 transmits a pulse signal or a burst signal of ultrasound of several MHz to several tens of MHz from a transmission section toward the measurement target blood vessel. Then, reflected waves from a front wall and a back wall of a measurement target blood vessel are received by a reception section and the blood vessel diameter is measured at the measurement target blood vessel from a reception time difference of the reflected waves of the front wall and the back wall.

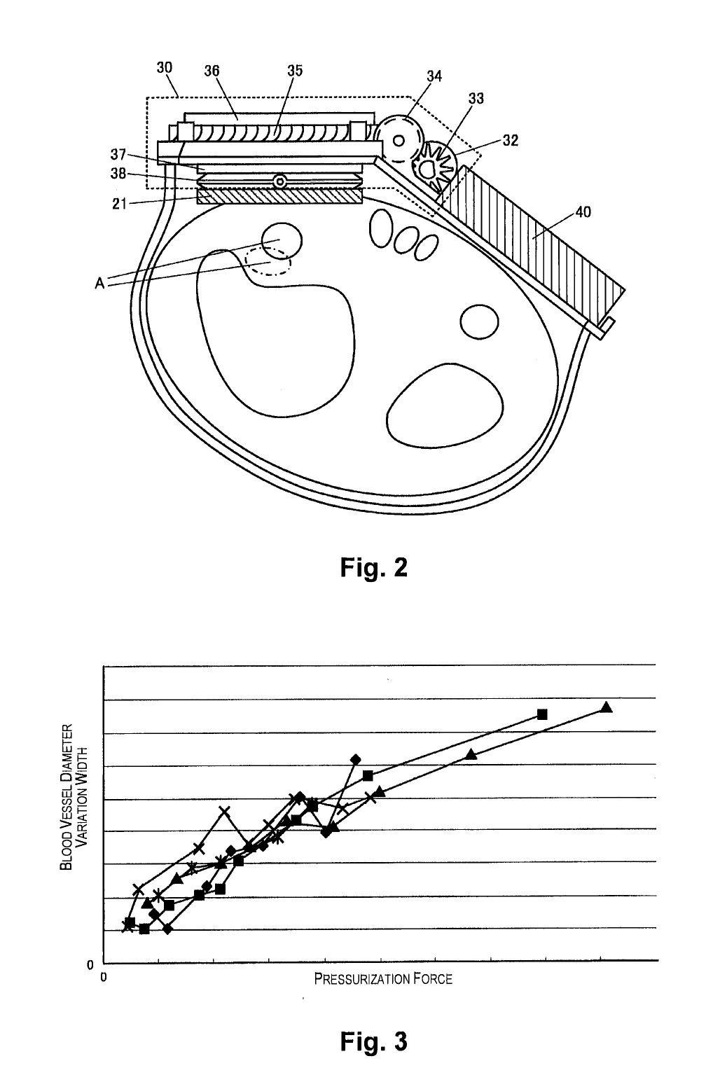

As shown in FIG. 2, the pressurizing section 30 is a pressurizing mechanism which is configured to have a cylindrical cam mechanism and is configured to be disposed directly above the ultrasound sensor 21. The pressurizing section 30 performs pressurization from directly above the ultrasound sensor 21 in a state where the ultrasound blood pressure monitor 1 is mounted on the measurement target portion and the body surface which is in contact with the ultrasound sensor 21 is pressurized.

Here, although omitted in the diagrams, a control substrate for comprehensively controlling the device is built into the body section of the ultrasound blood pressure monitor 1. A microprocessor, a memory, a circuit which is related to the sending and receiving of ultrasound, a built-in battery, and the like are mounted in the control substrate.

FIG. 1B is a diagram illustrating a state where the ultrasound blood pressure monitor 1 is mounted onto the left wrist of the patient. As shown in FIG. 1B, the ultrasound blood pressure monitor 1 is mounted onto the wrist of the patient with a positioning so that the body section is directed to the inner side of the wrist. At this time, the second portion 1B where the sensor section 20 is provided is mounted so as to come to a position on the thumb side of the wrist of the patient. This is so that the sensor section 20 is positioned directly above this with the measurement target blood vessel as the radial artery where there is flow to the thumb side of the wrist.

FIG. 2 is a cross sectional diagram of a state where the ultrasound blood pressure monitor 1 is mounted on a wrist. Here, an external cover of the body section is omitted in the diagram so that it is possible to recognize the inner portion of the apparatus in FIG. 2.

In the pressurizing section 30, a transmission gear 33 is rotated and a worm gear 34 which screws together with the transmission gear 33 is rotated due to an electromagnetic motor 32 being rotated by receiving electric power from a power source section 40. Accompanying this, the protrusion amount of the cylindrical cam mechanism 36 is controlled due to the rotating of a worm gear wheel 35 and there is a formation where a pressurizing plate 37 and a sensor water bag (pressure sensor) 38 are pushed out to the wrist side due to an increase in the protrusion amount.

The ultrasound sensor 21 is provided directly below the sensor water bag 38. The ultrasound sensor 21 is pressurized toward the body surface at the wrist due to the pushing out of the sensor water bag 38. That is, the ultrasound sensor 21 pressurizes the body surface directly above a radial artery A. The radial artery A changes shape due to the pressurization. In the embodiment, there is a characteristic where ultrasound is transmitted from the ultrasound sensor 21 in a state where the shape of the radial artery A has changed and the blood vessel diameter of the radial artery A is calculated based on the reflected wave.

### 2. Principles

FIG. 3 is a graph illustrating the extent to which the blood vessel diameter variation width changes in accompaniment with a pulsation. Experiments, where the blood vessel diameter variation width accompanying a pulsation is measured, were performed while changing the pressurization force with regard to the radial artery with a plurality of patients as targets. In FIG. 3, the horizontal axis is pressurization force and the vertical axis is blood vessel diameter variation width. The plotting formations are different for each patient. From the experiments, it was understood that the blood vessel diameter variation width increases in accompaniment with an increase in the pressurization force.

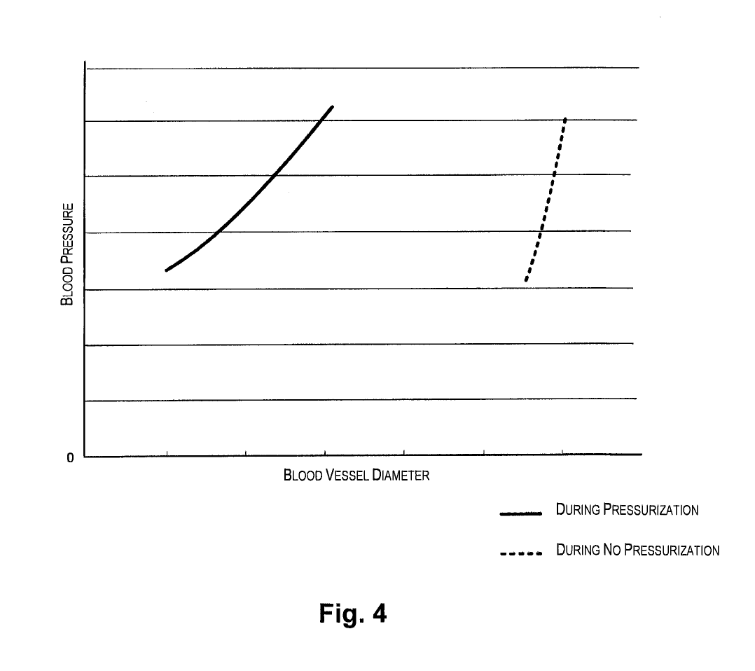

FIG. 4 is a diagram illustrating the extent to which correlation characteristics of blood vessel diameter and blood pressure change when there is pressurization and no pressurization with regard to the radial artery. In the embodiment, the correlation characteristics of blood vessel diameter “D” and the blood pressure “P” are approximated using a non-linear correlation formula which is expressed by the following formula (1).

P=Pd·exp[β(D/Dd−1)] (1)

Here, β=In(Ps/Pd)/(Ds/Dd−1)

In formula (1), “Ps” is contraction phase blood pressure (maximum blood pressure) and “Pd” is extension phase blood pressure. In addition, “Ds” is contraction phase blood vessel diameter which is the blood vessel diameter when there is the contraction phase blood pressure and “Dd” is extension phase blood vessel diameter which is the blood vessel diameter when there is the extension phase blood pressure. In addition, “β” is a blood vessel elasticity index called a stiffness parameter.

The graph in FIG. 4 is a graph which is formed in accordance with formula (1), the curve shown with the dotted line shows the correlation formula of blood vessel diameter “D” and the blood pressure “P” when there is no pressurization, and the curve shown with the solid line shows the correlation formula of blood vessel diameter “D” and the blood pressure “P” when there is pressurization. When viewing the graph, it is understood that the slope of the curve is smaller when there is pressurization compared to when there is no pressurization. That is, the blood vessel diameter variation width is larger with regard to the same change in blood pressure when there is pressurization.

When specific numerical values are exemplified, the amount of variation in blood vessel diameter is approximately 200 [μm] with regard to a change in blood pressure of approximately 60 [mmHg] in a case where the body surface is pressurization by 50 [mmHg], compared to there being only a change in blood vessel diameter of approximately 50 [μm] with regard to a change in blood pressure of approximately 60 [mmHg] when there is no pressurization. Accordingly, when the degree of accuracy of blood vessel diameter measurement is the same, error in the blood vessel diameter measurement is “approximately ¼” when there is pressurization. As a result, it is possible to improve the degree of accuracy in blood pressure calculation.

Based on this insight, in the embodiment, a pressure, where the variation width of the blood vessel diameter accompanying a pulsation measured using ultrasound satisfies a predetermined threshold, is searched for by controlling the pressurization by the pressurizing section 30 so as to change. Then, the correlation formula of the blood vessel diameter and the blood pressure of the radial artery in a state of pressurization by the pressurizing section 30 with the pressure is found and stored in a storage section. During measurement of blood pressure, the blood pressure “P” of the patient is calculated using the blood vessel diameter “D”, which is measured using ultrasound in a state of controlling the pressurization so that the pressurizing section 30 pressurized with the pressure which is stored in the storage section, and the correlation formula which is stored in the storage section.

### 3. Functional Configuration

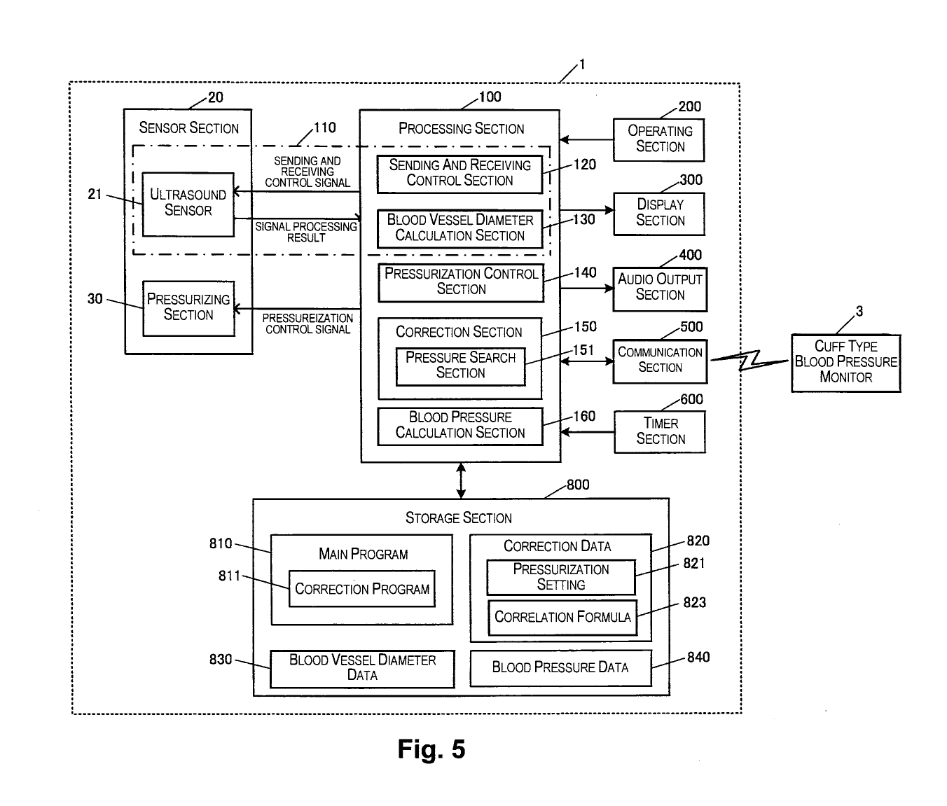

FIG. 5 is a block diagram illustrating an example of a functional configuration of the ultrasound blood pressure monitor 1. The ultrasound blood pressure monitor 1 is configured to have the sensor section 20, a processing section 100, an operating section 200, a display section 300, an audio output section 400, a communication section 500, a timer section 600, and a storage section 800.

The sensor section 20 is provided with the ultrasound sensor 21 and the pressurizing section 30. The ultrasound sensor 21 is an ultrasound sending and receiving section and is configured to have an ultrasound sending and receiving circuit. The ultrasound sending and receiving circuit sends and receives ultrasound by, for example, switching between an ultrasound transmission mode and an ultrasound reception mode with a time division method in accordance with a sending and receiving control signal which is output from the sending and receiving control section 120.

The sending and receiving circuit is configured to have an ultrasound oscillation circuit which generates a pulse signal with a predetermined frequency, a transmission delay circuit which delays the pulse signal which has been generated, and the like as a transmission configuration. In addition, the sending and receiving circuit is configured to have a reception delay circuit which delays a reception signal, a filter which extracts a predetermined frequency component from the reception signal, an amplifier which amplifies the reception signal, and the like as a configuration for reception.

The processing section 100 is a control apparatus and a computation apparatus which comprehensively controls each section of the ultrasound blood pressure monitor 1 and is configured to have, for example, a microprocessor such as a CPU (Central Processing Unit) or a DSP (Digital Signal Processer), an ASIC (Application Specific Integrated Circuit), and the like.

The processing section 100 has the sending and receiving control section 120, a blood vessel diameter calculation section 130, a pressurization control section 140, a correction section 150, and a blood pressure calculation section 160 as the main functional sections. Here, these functional section are only a description of one example and it is not necessary the case that all of these functional sections are essential configuration elements.

The sending and receiving control section 120 controls the sending and receiving of ultrasound by the ultrasound sensor 21. Specifically, a sending and receiving control signal is output with regard to the ultrasound sensor 21 and control of switching between the transmission mode and the reception mode described above is performed

The blood vessel diameter calculation section 130 calculates the blood vessel diameter of the measurement target blood vessel based on signal processing results which are input from the ultrasound sensor 21. Specifically, the blood vessel diameter of the measurement target blood vessel is calculated by detecting a reception time difference of the reflected ultrasound waves of the front wall and the back wall of the measurement target blood vessel.

In the embodiment, a blood vessel diameter measurement section 110 which measures the blood vessel diameter of the artery which is the measurement target (the radial artery) is configured by the ultrasound sensor 21, the sending and receiving control section 120, and the blood vessel diameter calculation section 130.

The pressurization control section 140 controls the pressurization by the pressurizing section 30 with regard to the measurement target portion. Specifically, the measurement target portion is pressurized by a predetermined pressurization force by outputting a pressurization control signal with regard to the pressurizing section 30. In the embodiment, a pressurization setting 821 of the pressurizing section 30 is decided on in a correction process which is performed by the correction section 150.

The correction section 150 performs correction of the ultrasound blood pressure monitor 1 at an initial correction after the power is turned on or a predetermined correction timing in accordance with a correction program 811 which is stored in the storage section 800. The correction section 150 has a pressure search section 151 which carries out a function of a first pressure search section which searches for a pressure where the variation width of the blood vessel diameter accompanying a pulsation, which is measured by the blood vessel diameter measurement section 110, satisfies a predetermined condition.

The blood pressure calculation section 160 calculates the blood pressure of the patient using the blood vessel diameter which is measured by the blood vessel diameter measurement section 110 and the correlation formula which indicates the correlation characteristics of blood vessel diameter and blood pressure. In the embodiment, a correlation formula 823 is decided on in the correction process which is performed by the correction section 150.

The operating section 200 is an input apparatus which is configured to have a button switch and the like and a signal of a button which has been pressed is output to the processing section 100. Due to the operation of the operating section 200, the input of various types of instructions such as an instruction for the starting of measurement of blood vessel diameter is carried out. The operating section 200 is equivalent to the operation button 12 in FIG. 1.

The display section 300 is a display apparatus which is configured to have an LCD (Liquid Crystal Display) or the like and performs various types of display based on a display signal which is input from the processing section 100. Information such as the blood pressure which is calculated by the blood pressure calculation section 160 is displayed in the display section 300. The display section 300 is equivalent to the liquid crystal display unit 13 in FIG. 1.

The audio output section 400 is an audio output apparatus which performs various types of audio output based on an audio output signal which is input from the processing section 100. The audio output section 400 is equivalent to the speaker 14 in FIG. 1.

The communication section 500 is a communication apparatus for sending and receiving information which is used in the apparatus to and from an external information processing apparatus in accordance with the control of the processing section 100. As the communication method of the communications section 500, it is possible to apply various methods such as a format where a cable which complies with a predetermined communication standard is connected in a wired manner, a format where there is connection via an intermediate apparatus which is also used as a recharger referred to as a cradle, a format where wireless communication is performed using short-distance wireless communication, or the like. In the embodiment, the communication section 500 performs the sending and receiving of data with the cuff type blood pressure monitor 3 using short-distance wireless communication.

The timer section 600 is a timer apparatus which is configured to have a crystal oscillator, which is formed by a crystal resonator and an oscillator circuit, or the like and measures time. The time measuring of the timer section 600 is output at any time to the processing section 100.

The storage section 800 is configured to have a storage apparatus such as a ROM (Read Only Memory), a flash ROM, a RAM (Random Access Memory), or the like. The storage section 800 stores a system program of the ultrasound blood pressure monitor 1, various types of programs for realizing each of the functional sections of the sending and receiving control function, the blood vessel diameter measurement function, and the blood pressure calculation function, data, and the like. In addition, there is a work area which temporarily stores processing data of various types of processing, processing results, and the like.

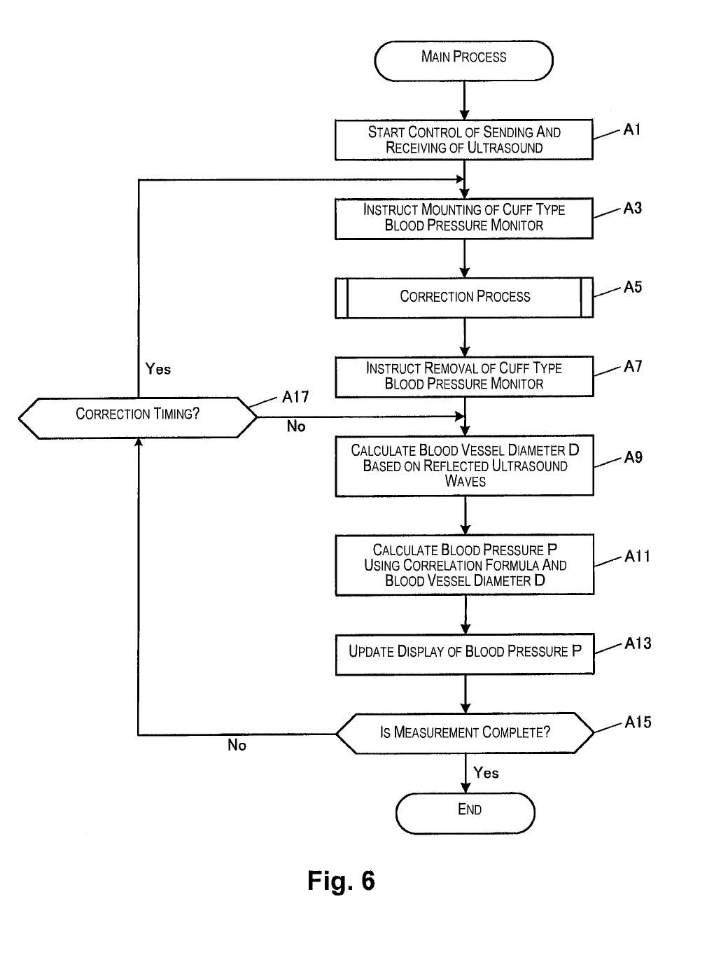

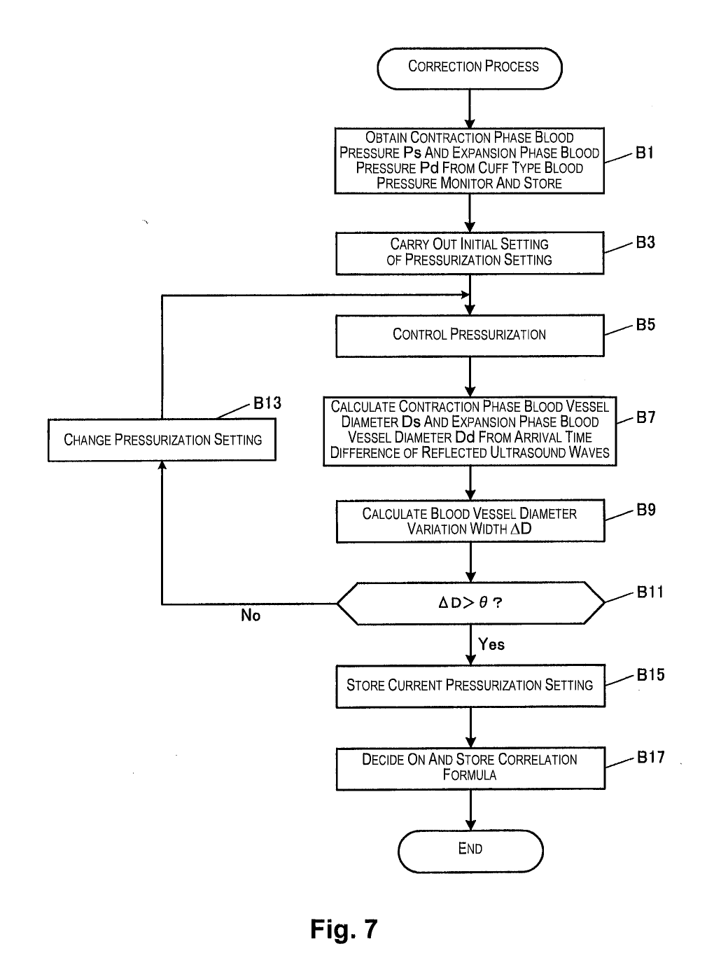

A main program 810, which is read out by the processing section 100 and is executed as a main process (refer to FIG. 6), is stored in the storage section 800 as a program. The main program 810 includes the correction program 811 which is executed as the correction process (refer to FIG. 7) as a subroutine. This process will be described later in detail using a flow chart.

In addition, correction data 820, the blood vessel diameter data 830, and blood pressure data 840 are stored in the storage section 800 as data.

The correction process data 820 is data where the correction results from the correction section 150 are stored, and the pressurization setting 821 which is the setting of the pressure which is searched for by the pressure search section 151 and the correlation formula 823 which sets the correlation characteristics of blood vessel diameter and blood pressure are included in this.

The blood vessel diameter data 830 is data where the blood vessel diameter of the measurement target blood vessel which is measured by the blood vessel diameter measurement section 110 is stored. The contraction phase blood vessel diameter and the extension phase blood vessel diameter are included in this.

The blood pressure data 840 is data where the blood pressure of the measurement target blood vessel which is calculated by the blood pressure calculation section 160 is stored. The contraction phase blood pressure and the extension phase blood pressure are included in this.

### 4. Process Flow

FIG. 6 is a flow chart illustrating the flow of the main process which is executed by the processing section 100 in accordance with the main program 810 which is stored in the storage section 800.

To begin with, the sending and receiving control section 120 starts sending and receiving control of the ultrasound from the ultrasound sensor 21 (step A1). Then, the processing section 100 performs an instruction for mounting the cuff type blood pressure monitor 3 with regard to the patient (step A3).

The instruction for mounting the cuff type blood pressure monitor 3 can be realized by displaying a message which prompts a mounting instruction on the display section 300 or can be realized by there being audio output of voice guidance or a predetermined generation of sound which prompts a mounting instruction from the audio output section 400. The patient can be notified by controlling a predetermined lamp to light up or to flash.

Next, the processing section 100 performs the correction process in accordance with the correction program 811 which is stored in the storage section 800 (step A5).

FIG. 7 is a flow chart illustrating the flow of the correction process. To being with, the correction section 150 acquires the contraction phase blood pressure “Ps” and the extension phase blood pressure “Pd” from the cuff type blood pressure monitor 3 via the communication section 500 and stores the contraction phase blood pressure “Ps” and the extension phase blood pressure “Pd” in the storage section 800 (step B1). Then, the correction section 150 carries out the initial setting of the pressurization setting 821 (step B3). Specifically, an initial value of, for example, 10 [mmHg] is set as the pressurization force of the pressurizing section 30.

Next, the pressure search section 151 makes the pressurization control section 140 execute control of the pressurization of the pressurizing section 30 (step B5). The blood vessel diameter calculation section 130 calculates the blood vessel diameter of the measurement target blood vessel from an arrival time difference of the reflected waves from the front wall and the back wall of the measurement target blood vessel (step B7). At this time, each of the contraction phase blood vessel diameter “Ds” and the extension phase blood vessel diameter “Dd” are calculated by tracking the variation in the blood vessel diameter accompanying a pulsation.

Next, the pressure search section 151 calculates a blood vessel diameter variation width “ΔD” by subtracting the contraction phase blood vessel diameter “Ds” which is calculated in step B7 from the extension phase blood vessel diameter “Dd” (step B9). Then, the pressure search section 151 determines whether the blood vessel diameter variation width “ΔD” exceeds a predetermined variation width threshold “θ” (step B11). It is possible to set the variation width threshold “θ” based on the relationship between the pulse pressure (the difference in the contraction phase blood pressure and the extension phase blood pressure) and the variation width of the blood vessel diameter accompanying a pulsation.

In a case where it is determined in step B11 that the blood vessel diameter variation width “ΔD” does not exceed the variation width threshold “θ” (step B11; No), the pressure search section 151 changes the pressurization setting 821 (step B13). For example, a pressure where 10 [mmHg] is added to the setting value of the current pressurization force is set as a new pressurization force. Then, the pressure search section 151 returns the process to step B5.

On the other hand, in a case where it is determined in step B11 that the blood vessel diameter variation width “ΔD” does exceed the variation width threshold “θ” (step B11; Yes), the pressure search section 151 stores the current pressurization setting 821 in the correction data 820 in the storage section 800 (step B15). The series of processes from step B3 to step B15 are equivalent to a pressure search process which is performed by the pressure search section 151.

Next, the correction section 150 decides on the correlation formula 823 of formula (1) using the contraction phase blood pressure “Ps” and the extension phase blood pressure “Pd” which are acquired from the cuff type blood pressure monitor 3 in step B1 and the latest values for the contraction phase blood vessel diameter “Ds” and the extension phase blood vessel diameter “Dd” which are acquired in step B7, and the correlation formula is stored in the correction data 820 (step B17). Then, the processing section 100 terminates the correction process.

Returning to the main process of FIG. 6, after the correction process has been completed, the processing section 100 performs an instruction for removal of the cuff type blood pressure monitor 3 with regard to the patient (step A7). It is possible for the instruction for the removal of the cuff type blood pressure monitor 3 to be performed with regard to the patient using a technique which is similar to the instruction for the mounting of the cuff type blood pressure monitor in step A3.

Next, the processing section 100 calculates the blood vessel diameter “D” based on the ultrasound reflected wave and stores the blood vessel diameter “D” in the blood vessel diameter 830 of the storage section 800 (step A9). Then, the processing section 100 calculates the blood pressure “P” using the correlation formula 823 which is stored in the storage section 800 and the blood vessel diameter “D” which is calculated in step A9 and stores the blood pressure “P” in the blood pressure data 840 of the storage section 800 (step A11). The processing section 100 updates the display of the display section 300 using the blood pressure “P” which has been calculated (step A13).

Next, the processing section 100 determines whether the measurement of blood pressure is complete (step A15), and in a case where it is determined that the measurement has not yet been completed (step A15; No), it is determined whether it is a correction timing (step A17). As the correction timing in this case, the setting of various timings are possible. For example, a case where a measurement timing of the timer section 600 becomes a timing which is set in advance (for example, 8 o'clock in the morning) can be determined to be the correction timing.

If it is determined that it is the correction timing (step A17; Yes), the processing section 100 returns the process to step A3. Then, the correction process using the cuff type blood pressure monitor 3 is executed again. In addition, if it is determined that it is not the correction timing (step A17; No), the processing section 100 returns the process to step A9. Then, the calculation of the blood pressure in continued.

On the other hand, in a case where it is determined in step A15 that the measurement has been completed (step A15; Yes), the processing section 100 terminates the main process.

### 5. Action Effects

In the ultrasound blood pressure monitor 1, the blood vessel diameter measurement section 110 measures the blood vessel diameter of the radial artery which is the measurement target based on the reception results of the ultrasound from the ultrasound sensor 21. In addition, the pressurizing section 30 adds a pressure from the body surface so that the radial artery is pressed. Then, the correlation formula 823, which expresses the relationship between blood vessel diameter and blood pressure of the radial artery under pressurization by the pressurizing section 30, is found and stored in the storage section 800. Then, the blood pressure calculation section 160 calculates the blood pressure by controlling the pressurization operation of the pressurizing section 30 using the blood vessel diameter which is measured under pressurization and the storage data in the storage section 800.

As is described in the principles, the variation width of the blood vessel diameter accompanying a pulsation increases with regard to the same change in blood pressure when the pressure is added from the body surface so that the radial artery is pressed compared to when there is no pressurization. Accordingly, it is possible to reduce the effect of error in the blood vessel diameter measurement by performing pressurization. That is, it is possible to improve the degree of accuracy in blood pressure calculation by increasing the change in blood pressure with regard to the variation in blood vessel diameter due to pressurization.

In the embodiment, the pressure search section 151 searches for the pressure, where the variation width of the blood vessel diameter accompanying a pulsation which is measured by the blood vessel diameter measurement section 110 satisfies the predetermined condition, by controlling the pressure due to the pressurizing section 30 so as to change. In detail, the pressure is searched for so that the variation width of the blood vessel diameter accompanying a pulsation which is measured by the blood vessel diameter measurement section 110 exceeds the predetermined variation width threshold which is set based on the relationship between the pulse pressure and the variation width. Due to this, it is possible to adjust the pressure which is used in pressurization by the pressurizing section 30.

The storage section 800 stores the correction formula 823 which indicates the relationship between blood vessel diameter and blood pressure of the artery in a state of pressurization with the pressure which is searched for by the pressure search section 151. Then, the blood pressure calculation section 160 calculates the blood pressure of the patient in a state where the pressurization of the pressurizing section 30 is controlled so that there is pressurization with the pressure which is searched for by the pressure search section 151. The relationship between blood vessel diameter and blood pressure, in a case where control of the pressurization by the pressurizing section 30 was performed with the pressure which is searched for by the pressure search section 151, is stored as data, and it is possible to correctly calculate the blood pressure of the patient by using the blood vessel diameter which is measured in a state of pressurization with the pressure which is the same as the pressure which is searched for by the pressure search section 151 and the correlation formula 823 which is stored in the storage section 800.

### 6. Modified Example

Naturally, the embodiments where it is possible for the invention to be applied are not limited to the embodiment described above and appropriate changes are possible in a scope which does not depart from the gist of the invention. Below, modified examples will be described.

### 6-1. Measurement Target Artery

In the embodiment described above, the artery which is the measurement target is described as the radial artery in the wrist, but naturally, arteries other than this can be the artery which is the measurement target. Since the technique of the embodiment is particularly effective in a case where a blood vessel which is relatively tough is the measurement target, for example, a limb artery other than the radial artery can be the measurement target artery.

### 6-2. Method for Measuring Blood Vessel Diameter

In the embodiment described above, the method for measuring the blood vessel diameter is described as a measuring method which uses ultrasound, but naturally, the method for measuring the blood flow speed is not limited to this. For example, a technique can be adopted where light of a predetermined wavelength is irradiated from a light emitting element toward the artery which is the measurement target and measurement of blood vessel diameter is performed based on the reflected light.

### 6-3. Ultrasound Blood Pressure Monitor

In the embodiment described above, the ultrasound blood pressure monitor 1 which is used by being mounted onto the wrist of the patient is described as an example, but there can be an ultrasound blood pressure monitor which is used by, for example, being wrapped around the upper arm. In this case, for example, a configuration is possible where the blood pressure is measured by mounting the ultrasound blood pressure monitor on the upper arm of one arm and the blood pressure is measured by mounting the cuff type blood pressure monitor on the upper arm of the other arm. A specific embodiment in this case (referred to below as a second embodiment) will be described below.

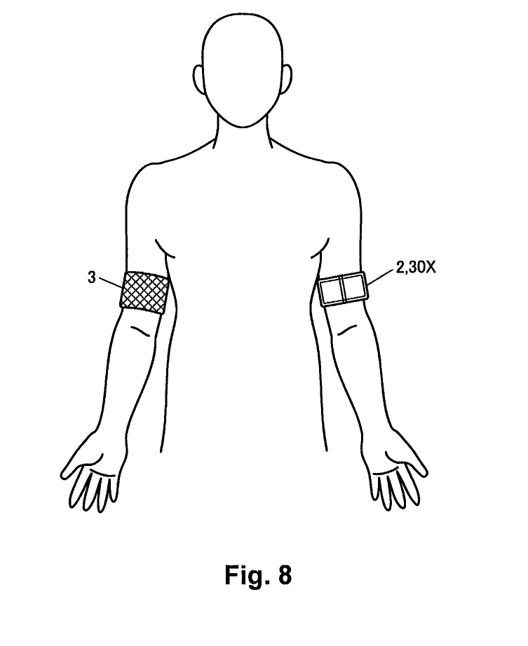

FIG. 8 is a diagram illustrating an example of a configuration of a blood pressure measurement system according to the second embodiment. In the blood pressure measurement system, an ultrasound blood pressure monitor 2 is mounted on the upper arm of one arm of the patient and the cuff type blood pressure monitor 3 is mounted on the upper arm of the other arm of the patient. The ultrasound blood pressure monitor 2 has a configuration which is basically the same as the ultrasound blood pressure monitor 1, but is configured to be provided with a cuff band and a pressurizing section 30X, which has a pressurizing mechanism for pressurization of the upper arm by sending air into the cuff band, instead of the pressurizing section 30 of the ultrasound blood pressure monitor 1 and so that it is possible to pressurize the upper arm of the patient in a uniform manner.

In the second embodiment, the ultrasound blood pressure monitor 2 measures the blood vessel diameter of the upper arm artery using ultrasound in a state where the upper arm is pressurized with a pressurization force “Po” for correction (referred to below as “correction pressurization force”). Since there are changes in a difference in the inner and outer pressure which is applied to the blood vessel measured using cuff pressure, the value of the stiffness parameter “β” is calculated in accordance with formula (2) using the blood vessel diameter which is measured and the blood pressure which is measured using the cuff type blood pressure monitor 3.

β=In[(Ps/Po)/(Pd−Po)]/(Ds/Dd−1) (2)

In addition, during normal measurement after correction, the ultrasound blood pressure monitor 2 measures the blood vessel diameter of the upper arm artery using ultrasound in a state of pressurization of the upper arm under a pressurization force “Po′” for normal measurement (which is described below as “normal measurement pressurization force”). Then, the blood pressure is calculated from the blood vessel diameter of the upper arm artery in accordance with the correlation formula of formula (3).

P=Pd·exp[β(D/Dd−1)]+Po′ (3)

It is possible to ignore the effect of the change in shape of the blood vessel since the ultrasound blood pressure monitor 2 presses the upper arm artery of the patient in a uniform manner. As a result, it is not necessary for the correction pressurization force “Po” and the normal measurement pressurization force “Po′” to be the same pressure and the pressures can be arbitrary pressures. Accordingly, it is possible to increase the degree of accuracy in blood vessel diameter measurement, and subsequently, the degree of accuracy in blood pressure calculation by setting the normal measurement pressurization force “Po′” as a pressure so that it is possible to increase the blood vessel diameter variation width. The normal measurement pressurization force “Po′” can be, for example, approximately 10 mmHg to 50 mmHg, and more appropriately, approximately 20 mmHg to 30 mmHg.

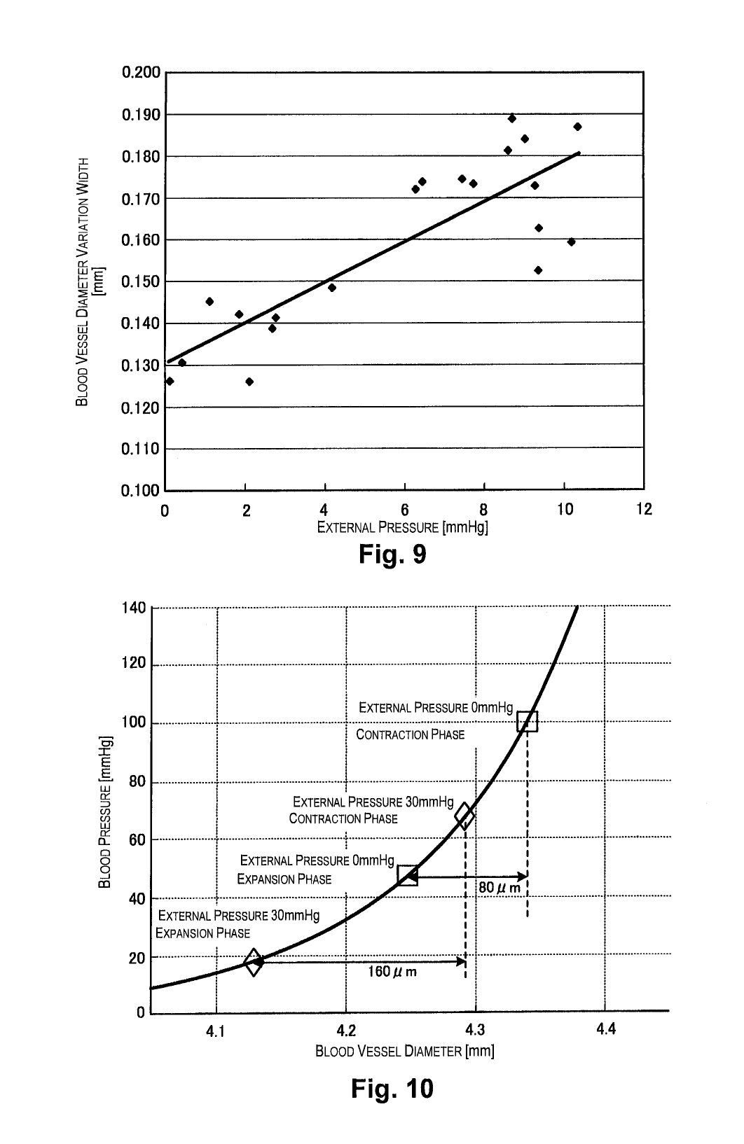

FIG. 9 is a diagram illustrating an example of changes in the variation width of the blood vessel diameter accompanying a pulsation in a case where the upper arm artery is pressurized. The horizontal axis is external pressure (the units are mmHg) and the vertical axis is blood vessel diameter variation width (the units are mm). From FIG. 9, it is understood that the blood vessel diameter variation width increases as the external pressure increases.

FIG. 10 is a diagram for describing effects of the blood pressure measurement method. The amounts of variation in blood vessel diameter are respectively shown by plotting in a case where the external pressure is 0 mmHg and in a case where the external pressure is 30 mmHg. The horizontal axis is blood vessel diameter (the units are mm) and the vertical axis is blood pressure (the units are mmHg). In addition, the curve which is shown by a solid line is a correlation formula which is given by formula (3). Compared to the amount of variation in blood vessel diameter of approximately 80 μm in a case where the external pressure is 0 mmHg, the amount of variation in blood vessel diameter is double at approximately 160 μm by applying the external pressure of 30 mmHg. Accordingly, the degree of accuracy of measurement is simply doubled since measurement is possible in a state where the size of the measurement target object is double.

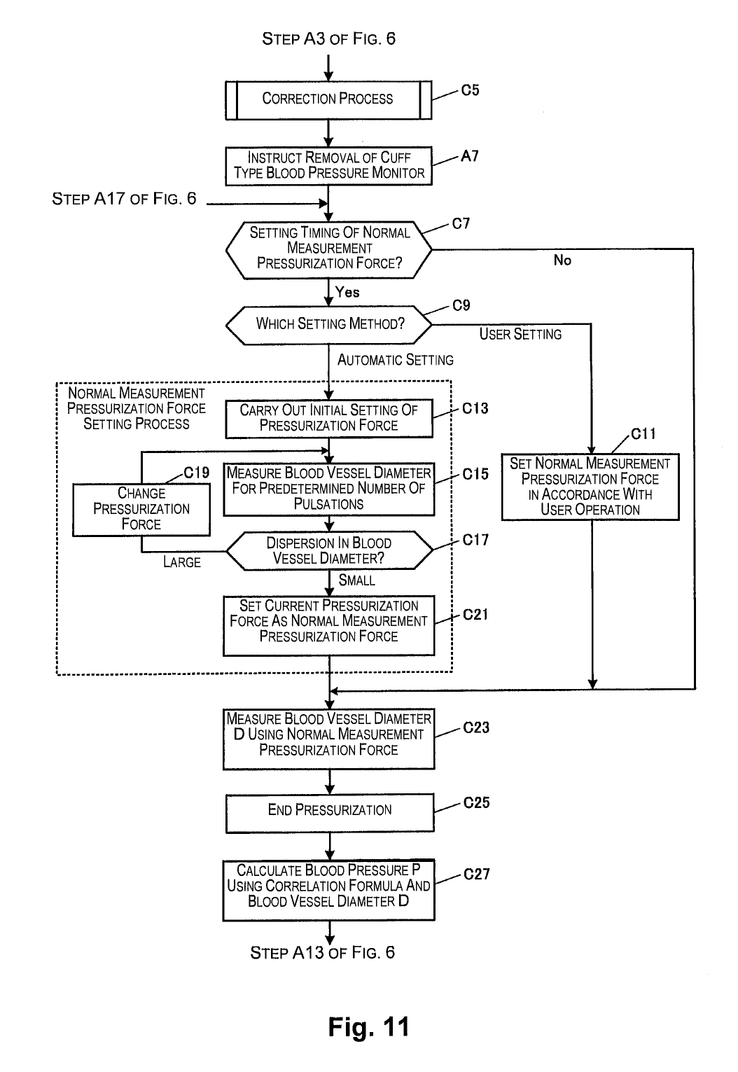

FIG. 11 is a flow chart where a portion of a second main process, which is executed by the processing section 100 of the ultrasound blood pressure monitor 2 in the second embodiment instead of the main process of FIG. 6, is extracted. Here, repetitive description is omitted by giving the same reference numerals to the same steps in the main process.

In the correction process, the value of the stiffness parameter “β” is calculated in accordance with formula (2) (step C5). After the instruction for the removal of the cuff type blood pressure monitor 3 has been carried out (step A7), the processing section 100 determines whether it is setting timing of the normal measurement pressurization force (step C7). For example, it is determined to be the setting timing in a case where the setting of the normal measurement pressurization force has been instructed by the user via the operating section 200 and in a case where a predetermined period of time (for example, one day) has elapsed from the performing of the last setting of the normal measurement pressurization force. In a case where it is determined to not be the setting timing (step C7; No), the process moves to step C23.

On the other hand, in a case where it is determined to not be the setting timing (step C7; Yes), the processing section 100 determines the setting method for the normal measurement pressurization force (step C9). In the setting method, there are the two types of an automatic setting and a user setting, and for example, the user selects either of the setting methods. In a case where the setting method which has been selected is the user setting (step C9; user setting), the processing section 100 sets the pressurization force which has been selected by the user as the normal measurement pressurization force (step C11). Then, the process moves to step C23. On the other hand, in a case where the setting method which has been selected is the automatic setting (step C9; automatic setting), the processing section 100 performs a normal measurement pressurization force setting process (steps C13 to C21).

Specifically, a predetermined initial value (for example, 10 mmHg) is initially set as the pressurization force (step C13) and the blood vessel diameter is measured for a predetermined number of pulsations (for example, 10 to 20 pulsations) using ultrasound in a state of pressurization with the pressurization force (step C15). Then, dispersion in the blood vessel diameter which has been measured is determined (step C17). It is possible to realize the determination of dispersion by, for example, calculating an average value and a standard deviation of the extension phase blood vessel diameter for a predetermined number of pulsations and by determining whether the standard deviation is less than a predetermined threshold. Here, the contraction phase blood vessel diameter can be used instead of the extension phase blood vessel diameter.

If it determined that the dispersion of the blood vessel diameter is large (step C17; large), the processing section 100 changes the pressurization force by addition of a predetermined value (for example, 5 mmHg) to the setting value of the current pressurization force or the like (step C19). Then, the process returns to step C15. On the other hand, if it determined that the dispersion of the blood vessel diameter is small (step C17; small), the current pressurization force is set as the normal measurement pressurization force (step C21). The series of processes of step C13 to C21 is equivalent to a process where there is searching for a pressure where the blood vessel diameter which is measured by the blood vessel diameter measurement section satisfies a predetermined stability condition, and in this case, the processing section 100 functions as a second pressure search section.

After step C21, the processing section 100 measures the blood vessel diameter D with the normal measurement pressurization force which has been set in the normal measurement pressurization force setting process (step C23). That is, the blood vessel diameter D is measured using ultrasound in a state where the pressurization of the pressurizing section 30 is controlled so as to pressurize with the pressure which has been searched for by the second search section. If the measurement is complete, the pressurization operation is halted (step C25). Then, the blood pressure P is calculated in accordance with formula (3) using the correlation formula which is found in the correction process and the blood vessel diameter D which has been measured (step C27). Then, the process moves to step A13 in FIG. 6.

Here, in the embodiment described above, it is not necessary for the ultrasound blood pressure monitor 1 and the cuff type blood pressure monitor 3 to perform measurement by being mounted on the same arm. By the ultrasound blood pressure monitor 1 and the cuff type blood pressure monitor 3 being mounted on different arms, the contraction phase blood pressure and the extension phase blood pressure can be measured using the cuff type blood pressure monitor 3 which is mounted on one of the arms and the contraction phase blood vessel diameter and the extension phase blood vessel diameter can be measured in a continuous manner using the ultrasound blood pressure monitor 1 which is mounted on the other arm.

In the same manner, in the second embodiment described above, it is not necessary for the ultrasound blood pressure monitor 2 and the cuff type blood pressure monitor 3 to perform measurement by being mounted on different arms. For example, the measurement can be performed by the ultrasound blood pressure monitor 2 being mounted on the upper arm of one of the arms and the cuff type blood pressure monitor 3 which is configured as a wrist type of blood pressure monitor being mounted on the wrist of the same arm.

In addition, in the second embodiment, the ultrasound blood pressure monitor 2 and the cuff type blood pressure monitor 3 can both have a pressurizing mechanism using a cuff. As a result, it is possible for the ultrasound blood pressure monitor 2 and the cuff type blood pressure monitor 3 to be configured integrally. In this case, the blood pressure is measured using an oscillometric method by pressurization so that there is expulsion of blood from the upper arm using the cuff during correction. In addition, the blood vessel diameter of the upper arm artery is measured using ultrasound in a state of pressurization of the upper arm with the correction pressurization force “Po”. Then, it is sufficient if the value of the stiffness parameter is calculated in accordance with formula (2) using the blood pressure measurement value and the blood vessel diameter measurement value.

In addition, it is possible to appropriately correct the correlation formula by carrying out processing to average each of the contraction phase blood vessel diameter and the extension phase blood vessel diameter which have been measured continuously and deciding on the correlation formula using the average value of the contraction phase blood vessel diameter and the average value of the extension phase blood vessel diameter. It is possible to further improve the degree of accuracy of blood pressure calculation by calculating the blood pressure using the correlation formula which has been found in this manner.

### 6-4. Correlation Characteristics

In the embodiment described above, the case of applying the correlation formula which is expressed by formula (1) as the correlation formula which expresses the correlation characteristics of blood vessel diameter and blood pressure is described as an example, but other than this, naturally, correlation formulae where blood vessel diameter and blood pressure are approximated using a linear relationship and correlation formulae where blood vessel diameter and blood pressure are approximated using a non-linear relationship other than formula (1) can be applied.

In addition, naturally, it is not necessary for data on the correlation characteristics which is stored in the storage section to be data on the correlation formula and such can be data where the correlation characteristics of blood vessel diameter pressure and blood pressure are set in a table format (a lookup table).

### 6-5. Correction Timing

In the embodiment described above, there is description where the correction process is performed at the timing which is an initial time of blood pressure measurement or is a timing which has been decided, but it is possible for the correction timing to be arbitrarily set. For example, there are cases where the shape of the measurement target blood vessel of the patient changes due to a rapid change in air temperature. Therefore, air temperature during the measurement of blood pressure can be stored and the correction process can be performed with a timing, where a difference in temperature of air temperature during the previous measurement and air temperature during the current measurement exceeds a predetermined threshold, as the correction timing.

### 6-6. Communication Method

In addition, in the embodiment described above, the communication method of the ultrasound blood pressure monitor 1 and the cuff type blood pressure monitor 3 is set as wireless communication, but can be wired communication by connection using a cable. In addition, the measurement of blood pressure can be performed by the patient using the cuff type blood pressure monitor 3 and the measurement value can be input by hand into the ultrasound blood pressure monitor 1 by the patient.

What is claimed is:

1. A blood pressure measurement apparatus comprising:
a blood vessel diameter measurement section which measures blood vessel diameter of an artery which is a measurement target;
a pressurizing section which adds pressure from a body surface so that the artery is pressed;
a storage section which stores a relationship between blood vessel diameter and blood pressure of the artery under pressurization by the pressurizing section; and
a blood pressure calculation section which calculates blood pressure by controlling a pressurization operation of the pressuring section using the blood vessel diameter, which is measured by the blood vessel diameter measurement section under pressurization, and storage data in the storage section.

2. The blood pressure measurement apparatus according to claim 1, further comprising:
a first pressure search section which searches for a pressure by controlling the pressure due to the pressurizing section so as to change so that the variation width in the blood vessel diameter accompanying a pulsation, which is measured by the blood vessel diameter measurement section, satisfies a predetermined condition,
wherein the storage section stores the relationship between the blood vessel diameter and the blood pressure of the artery in a state of pressurization with the pressure which has been searched for by the first pressure search section, and
the blood pressure calculation section controls the pressurization operation of the pressurizing section so that there is pressurization with the pressure which is searched for by the first pressure search section.

3. The blood pressure measurement apparatus according to claim 2,
wherein the first pressure search section searches for a pressure where the variation width of the blood vessel diameter accompanying a pulsation, which is measured by the blood vessel diameter measurement section, exceeds a predetermined variation width threshold which is set based on the relation of pulse pressure and the variation width.

4. The blood pressure measurement apparatus according to claim 1,
wherein the blood pressure calculation section controls the pressurization operation of the pressurizing section so as to pressurize with an arbitrary pressure.

5. The blood pressure measurement apparatus according to claim 4, further comprising:
a second pressure search section which searches for a pressure by controlling the pressurization by the pressurizing section so as to change so that the blood vessel diameter which is measured by the blood vessel diameter measurement section satisfies a predetermined stability condition,
wherein the blood pressure calculation section controls the pressurization of the pressurizing section so that there is pressurization with the pressure which is searched for by the second pressure search section.

6. A blood pressure measurement method for a blood pressure measurement apparatus which is provided with a pressurizing section which adds pressure from a body surface so that an artery which is a measurement target is pressed and a storage section which stores the relationship between blood vessel diameter and blood pressure of the artery under pressurization by the pressurizing section, the method comprising:
measuring blood vessel diameter of the artery; and
calculating blood pressure by controlling a pressurization operation of the pressuring section using the blood vessel diameter under pressurization and storage data in the storage section.
